(19) United States
(12) Patent Application Publication
(10) Pub. No.: US 2025/0177658 A1
(43) Pub. Date: Jun. 5, 2025

(54) DEVICE FOR SEPARATING BUBBLES FROM INJECTABLE DRUGS

(71) Applicant: CHUNGMED Co., Ltd., Seoul (KR)

(72) Inventor: Hyun Hoon CHUNG, Seoul (KR)

(73) Assignee: CHUNGMED Co., Ltd., Seoul (KR)

(21) Appl. No.: 18/920,856

(22) Filed: Oct. 19, 2024

(30) Foreign Application Priority Data
Dec. 4, 2023 (KR) 10-2023-0173334

(51) Int. Cl. A61M 5/36; A61M 5/165

(52) U.S. Cl. CPC A61M 5/36; A61M 2005/1652; A61M 2005/1657

(57) ABSTRACT

The device for separating bubbles from an injectable drug is disclosed, which prevents the bubbles from entering a blood vessel when the injectable solution is delivered to a vein. This device improves the stability of the injectable solution when administered to a human body, and the device is easily added to a conventional infusion or injectable solution sets, thereby improving user convenience and efficiency.

### CROSS REFERENCE TO RELATED APPLICATION

Priority to Korean patent application number 10-2023-0173334 filed on Dec. 4, 2023 the entire disclosure of which is incorporated by reference herein, is claimed.

### BACKGROUND OF THE INVENTION

### Field of the Invention

The disclosure relates to a device for injecting only a drug into a human body by separating bubbles from the drug when injecting the drug into the human body.

### Description of the Related Art

Intravenous injection refers to a medical procedure for injecting a drug or liquid directly into a blood vessel. The intravenous injection has an advantage of quickly and effectively delivering an injectable solution into the body. The intravenous injection has been widely used in delivering a drug, a fluid, blood, a contrast medium, and the like liquids for emergency situations, appropriate treatment, and patient's condition diagnosis into the human body.

In relation to such a conventional intravenous injection technology, Korean Patent No. 1657694 has been disclosed. However, when there are bubbles in the injectable solution during conventional intravenous injection, the injectable solution may be delivered to the human body together with the bubbles. A small amount of air bubbles entering blood vessels during the intravenous injection may interfere with blood flow, and a large air bubble or a collection of several small bubbles may cause vascular occlusion. Accordingly, there is a need for technology in which the bubbles are separated from the injectable solution and prevented from being delivered to the human body during the intravenous injection.

### DOCUMENTS OF RELATED ART

(Patent Document) Korean Patent No. 1657694

### SUMMARY OF THE INVENTION

An aspect of the disclosure is to provide a device for preventing an injectable solution and bubbles from being injected together during conventional intravenous injection.

According to the disclosure, there is provided a device for separating bubbles from an injectable drug, including: an inlet port configured to couple with a first tube; an outlet port configured to couple with a second tube; a bubble separating portion provided between the inlet port and the outlet port, and having a passage cross-sectional area more enlarged than the passage cross-sectional area of the first tube; and a mesh portion provided in a passage of the inlet port or a passage of the bubble separating portion.

Meanwhile, the bubble separating portion may include a spherical, ellipsoidal, or cylindrical inner space.

Further, a flow direction in the inlet port and a flow direction in the outlet port may be parallel to each other.

Meanwhile, the bubble separating portion may be rotationally symmetrical with respect to a central axis in the flow direction.

Meanwhile, the mesh portion may have a honeycomb structure.

### BRIEF DESCRIPTION OF THE DRAWINGS

FIG. 1 is a perspective view of a device for separating bubbles from an injectable drug according to a first embodiment of the disclosure.

FIG. 2 is a cross-sectional view of the device for separating bubbles from an injectable drug according to the first embodiment of the disclosure.

FIG. 3 is a perspective view of a mesh portion.

FIG. 4 is a view showing an operating state of the device for separating s from an injectable drug according to the first embodiment of the disclosure.

FIG. 5 is a view showing a usage state of the device for separating bubbles from an injectable drug according to the first embodiment of the disclosure.

FIG. 6 is a cross-sectional view of a device for separating bubbles from an injectable drug according to a second embodiment of the disclosure.

FIG. 7 is a view showing an operating state of the device for separating bubbles from an injectable drug according to the second embodiment of the disclosure.

FIG. 8 is a cross-sectional view of a device for separating bubbles from an injectable drug according to a third embodiment of the disclosure.

FIG. 9 is a cross-sectional view of a device for separating bubbles from an injectable drug according to a fourth embodiment of the disclosure.

FIG. 10 is a cross-sectional view of a device for separating bubbles from an injectable drug according to a fifth embodiment of the disclosure.

FIG. 11 is a cross-sectional view of a device for separating bubbles from an injectable drug according to a sixth embodiment of the disclosure.

### DETAILED DESCRIPTION OF THE INVENTION

Below, a device for separating bubbles from injectable drugs according to an embodiment of the disclosure will be described in detail with reference to the accompanying drawings. In the following description, the names of components used may be referred to as other names in this art. However, these components may be considered as equivalent components in alternative embodiments if they are functionally similar or identical to each other. Further, the reference numerals of the components are merely given for the convenience of description. However, the components indicated by the reference numerals in the accompanying drawings are not limited by those shown therein. Likewise, if components are functionally similar or identical to each other even though they are partially modified in the drawings according to alternative embodiments, the components may be considered as the equivalent components. Further, when components are recognized as components that should be included at the level of those skilled in the art, they are not described. In addition, if it is obvious to those skilled in the art that components should be included, descriptions of the components will be omitted.

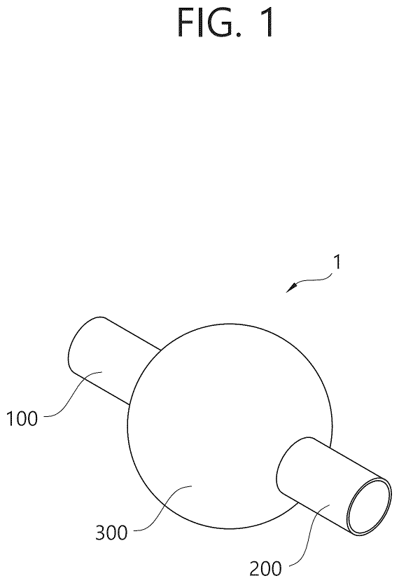

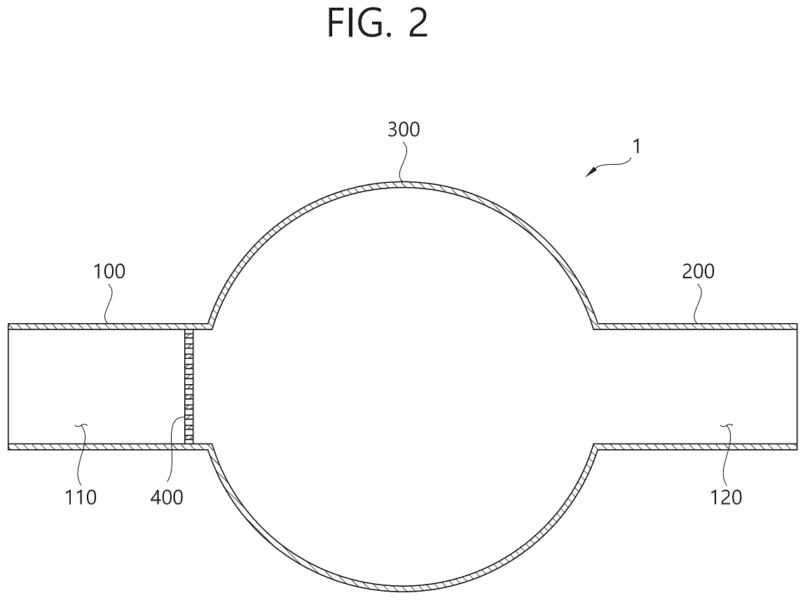

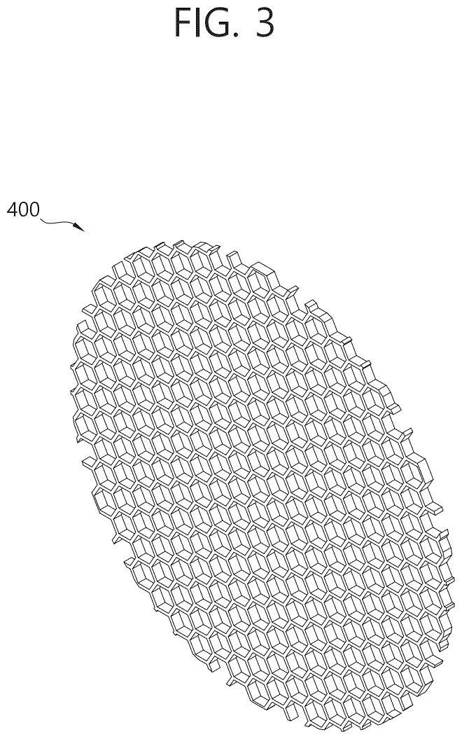

FIG. 1 is a perspective view of a device for separating bubbles from an injectable drug according to a first embodiment of the disclosure, FIG. 2 is a cross- sectional view of the device for separating bubbles from an injectable drug according to the first embodiment of the disclosure, and FIG. 3 is a perspective view of a mesh portion. Referring to FIGS. 1 to 3, a device 1 for separating bubbles 2000 from an injectable drug according to the first embodiment of the disclosure may include an inlet port 100, an outlet port 200, a bubble separating portion 300, and a mesh portion 400.

The inlet port 100 includes an inlet 110 through which an injectable solution 1000 such as a drug, a fluid, and a contrast medium flows in. The inlet port 100 is configured to couple with a first tube 10 connected to an external component that supplies an injectable solution 1000.

The outlet port 200 includes an outlet through which the injectable solution 1000 from a bubble separating portion 300 (to be described later) flows out. The outlet port 200 is configured to couple with a second tube 20 connected to a needle 30 inserted into a human body.

The bubble separating portion 300 is configured to separate the bubbles 2000 from the injectable solution 1000 flowing in from the inlet port 100 and allow only the pure injectable solution 1000 to flow out to the outlet port 200. The passage cross-sectional area of the bubble separating portion 300 may be larger than that of the first tube 10. As the passage cross-sectional area becomes larger, there are more opportunities to separate the bubbles 2000 from the injectable solution 1000. First, the bubbles 2000 have the characteristics of moving in the opposite direction to the gravity due to buoyancy, and thus move farther away from the center of the passage. Second, a sufficient time for the bubbles 2000 to move far away from the center of the passage may be secured because a flow speed decreases as the passage cross-sectional area becomes larger.

The bubble separating portion 300 may be provided with a spherical, ellipsoidal, or cylindrical inner space. Further, the bubble separating portion 300 may have an inner diameter more enlarged in a radial direction than those of the inlet port 100 and the outlet port 200. Therefore, the bubbles 2000 escaping from the center of the flow may stay in the inner space of the bubble separating portion 300. The inlet port 100 and the outlet port 200 may be concentrically configured, and the bubble separating portion 300 may be configured to be rotationally symmetrical with respect to the concentric axis. Therefore, the bubble separating portion 300 can maintain the function of separating the bubbles 2000 even though the angle of the bubble separating portion 300 is changed around the concentric axis.

The bubble separating portion 300 may be made of a light transmitting material. Therefore, the device 1 for separating the bubbles 2000 according to the disclosure allows medical staff to easily check the air bubbles trapped in the bubble separating portion 300.

The mesh portion 400 is configured to easily separate the bubbles 200 from the injectable solution 1000 including the air bubbles 2000. The mesh portion 400 may be placed adjacent to a connecting portion between an inlet port 100 and the bubble separating portion 300. The mesh portion 400 may include a plurality of pores formed in a flow direction.

The mesh portion 400 may for example have a honeycomb structure. Thus, the plurality of pores formed in the mesh portion 400 may have a hexagonal passage cross-sectional area.

When the mesh portion 400 has the honeycomb structure, the pressure and speed of the flow immediately after passing through the mesh portion 400 may have more uniform gradients under the same flow conditions. Therefore, the bubbles 2000 are more stably separated.

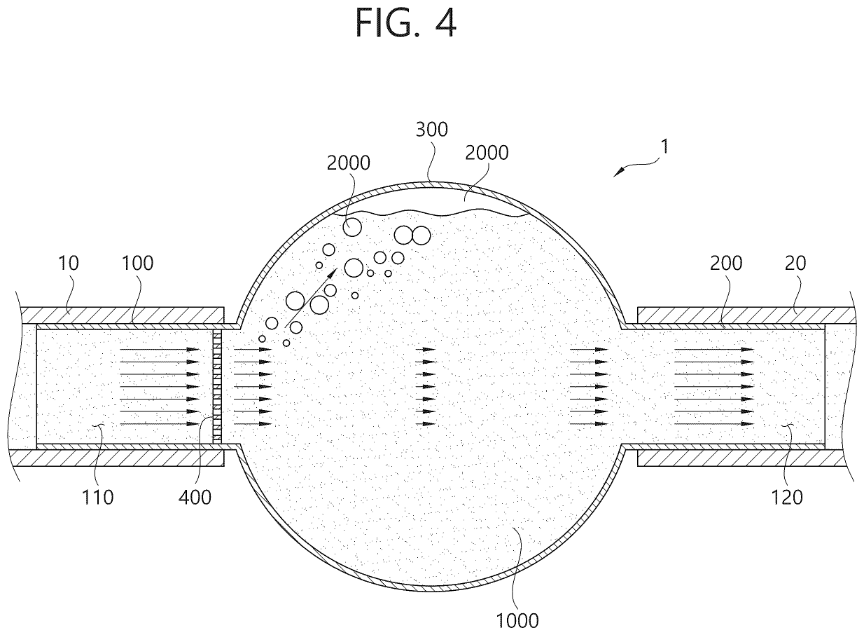

FIG. 4 is a view showing an operating state of the device 1 for separating bubbles 2000 from an injectable drug according to the first embodiment of the disclosure.

Referring to FIG. 4, the mesh portion 400 decreases the passage cross-sectional area in terms of the flow of the injectable solution 1000, and a short section after passing through the mesh portion 400 causes a turbulent flow. The turbulent flow has a dynamic effect on the bubbles 2000 in a direction different from a dominant flow direction in the first tube 10. Therefore, the bubbles 200 move upward more quickly based on the turbulent flow and the buoyancy.

Further, when the injectable solution 1000 enters the bubble separating portion 300 after passing through the mesh portion 400, the passage cross-sectional area increases and the flow speed decreases. In other words, when the injectable solution 1000 passes through the mesh portion 400, both the occurrence of the turbulent flow and the rapid decrease in the flow speed cause the bubbles 2000 to move upward more easily.

The bubble separating portion 300 stably separates the bubbles 2000 from the injectable solution 1000 and serves as a trap to prevent the bubbles 2000 from escaping as long as the inlet port 100 and the outlet port 200 are provided in an approximately horizontal direction. Meanwhile, the injectable solution 1000, from which the bubbles 2000 are separated, resumes being injected into a human body via the outlet of 210 of the outlet port 200.

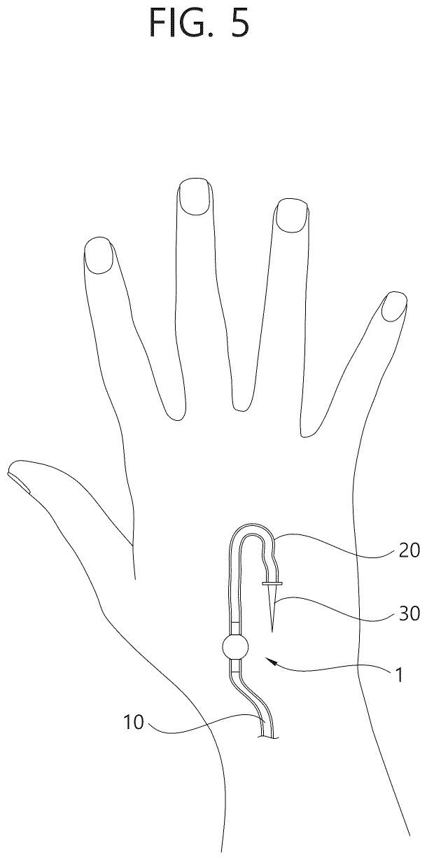

FIG. 5 is a view showing a usage state of the device 1 for separating bubbles 2000 from an injectable drug according to the first embodiment of the disclosure.

Referring to FIG. 5, the device 1 for separating bubbles 2000 from an injectable drug according to the first embodiment of the disclosure may be placed adjacent to the needle in a conventional infusion set including a plurality of components. In other words, the bubbles 2000 may be separated from the injectable solution 1000 in the final stage before the injectable solution 1000 is injected into the human body.

In this case, the bubble separating portion 300 has the rotationally symmetrical configuration and thus still maintains the function of separating the bubbles 2000 from the injectable solution 1000 even though a patient moves his/her hand and the hand is changed in angle.

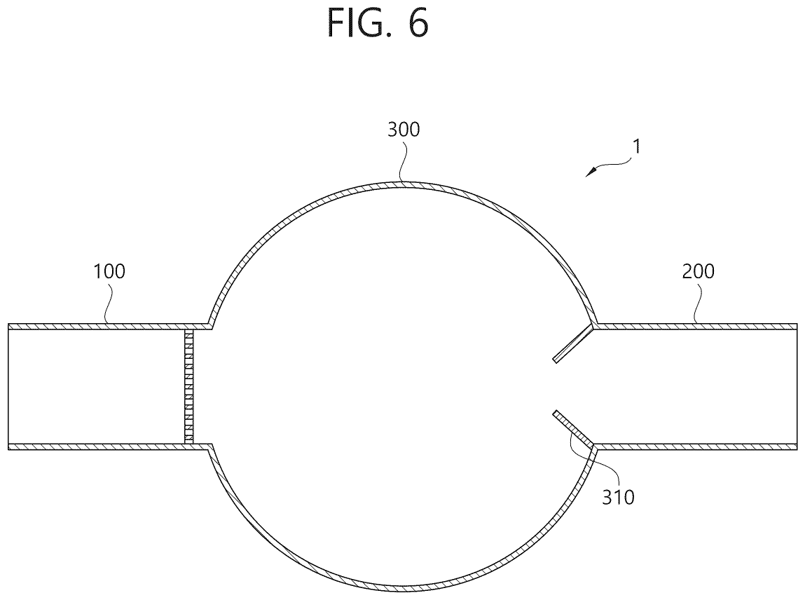

FIG. 6 is a cross-sectional view of a device 1 for separating bubbles 2000 from an injectable drug according to a second embodiment of the disclosure.

Referring to FIG. 6, the device 1 for separating bubbles 2000 from an injectable drug according to the second embodiment of the disclosure may further include an inner wall 310. The inner wall 310 is configured to prevent the bubbles 2000 collected inside the bubble separating portion 300 from escaping toward the outlet port 200. The inner wall 310 is formed extending to a predetermined length toward the inside of the bubble separating portion 300. For example, the inner wall 310 may be shaped like a funnel, an inner diameter of which increases outwards from the center of the bubble separating portion 300.

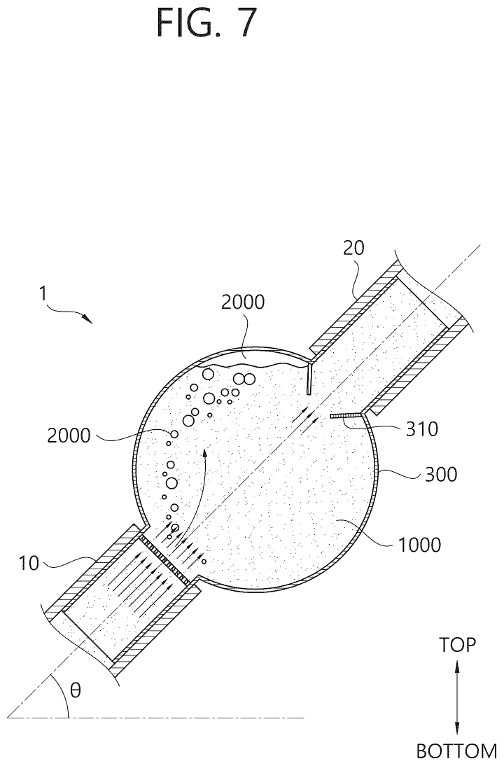

FIG. 7 is a view showing an operating state of the device 1 for separating bubbles 2000 from an injectable drug according to the second embodiment of the disclosure.

Referring to FIG. 7, the inner wall 310 in this embodiment is rotationally symmetrical with respect to the concentric c axis and thus still maintains the function of separating and collecting the bubbles 2000 even though a patient's hand is changed in angle 0 around the concentric axis. Further, according to this embodiment, the function of trapping the collected bubbles 2000 in the bubble separating portion 300 is strengthened even though the concentric axis of the first embodiment approaches the vertical.

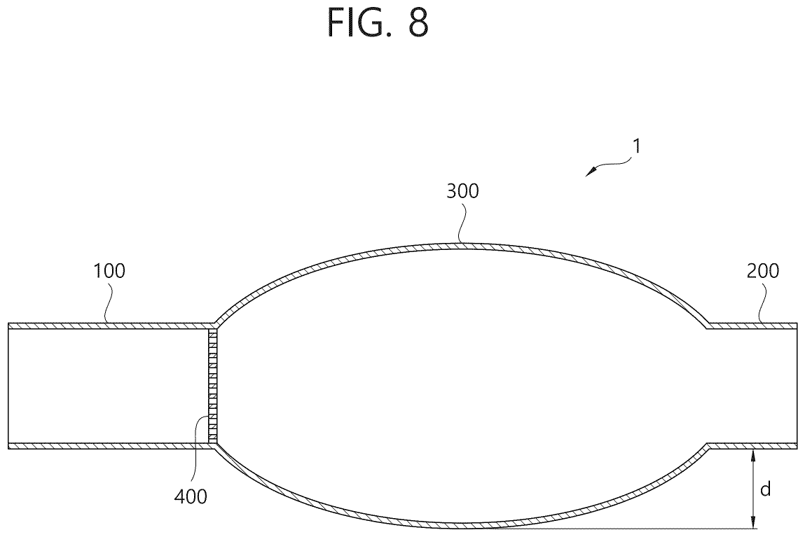

FIG. 8 is a cross-sectional view of a device 1 for separating bubbles 2000 from an injectable drug according to a third embodiment of the disclosure. Referring to FIG. 8, the device 1 for separating bubbles 2000 from an injectable drug according to the third embodiment of the disclosure may include the bubble separating portion 300 shaped like an ellipsoid. In the device 1 according to this embodiment, a space for collecting the bubbles 2000 is increased inside the bubble separating portion 300. Further, the outer diameter of the bubble separating portion 300 may be small even while the space for collecting the bubbles 2000 is secured inside the bubble separating portion 300. Therefore, a height difference d between the outer diameter of the bubble separating portion 300 and the inlet port 100 or the outlet port 200 is not large, thereby relieving a patient's discomfort even when the device 1 is actually attached to his/her body.

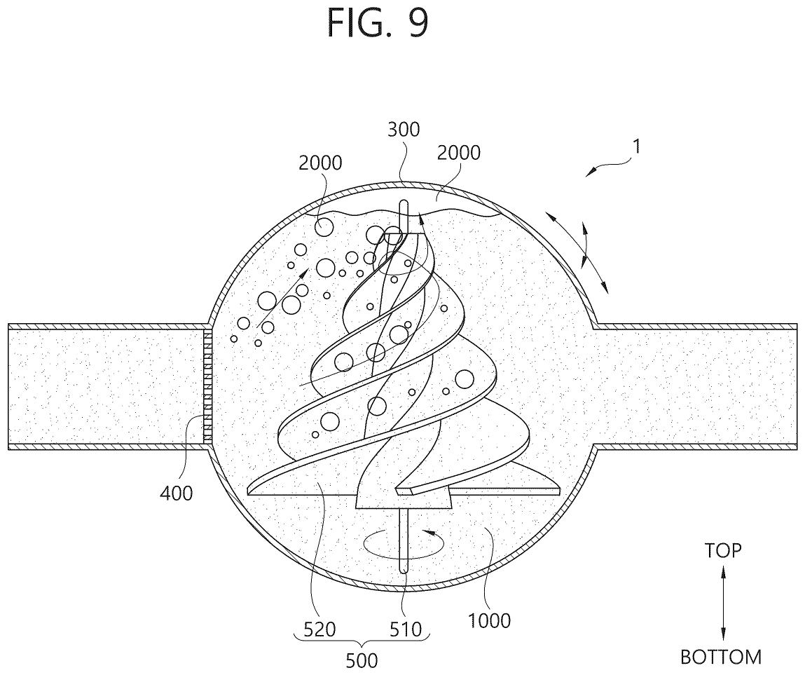

FIG. 9 is a cross-sectional view of a device 1 for separating bubbles 2000 from an injectable drug according to a fourth embodiment of the disclosure.

Referring to FIG. 9, the device 1 for separating bubbles 2000 from an injectable drug according to the fourth embodiment of the disclosure may include a rotor 500. The rotor 500 may be configured to rotate by the injectable solution 1000 when the injectable solution 1000 flows in the bubble separating portion 300. The rotor 500 may include a shaft 510 and a blade 520. The blade 520 includes a plurality of blades, has a width gradually decreasing in one direction, and spirally formed on the shaft 510. The shaft 510 may extend to a length slightly smaller than the inner diameter of the bubble separating portion 300.

The front half of the rotor 500 is rotated by the injectable solution 1000 with respect to the flow direction of the injectable solution 1000, but the second half affects the flow of the injectable solution 1000 based on the rotation of the rotor 500. The rotation of the rotor 500 causes the injectable solution 1000 passing through the second half to rotate and have angular momentum. In this case, when the injectable solution 1000 and the bubbles 2000 are mixed, the high-density injectable solution 1000 moves quickly outward, but the bubbles 2000 moves toward the center of the rotation. In other words, the rotation of the rotor 500 causes the bubbles 2000 to move toward not the outlet port but the central axis of the rotor 500 within a predetermined area. Ultimately, the rotation of the rotor 500 assists the bubbles 2000 in being separated and collected in the bubble separating portion 300.

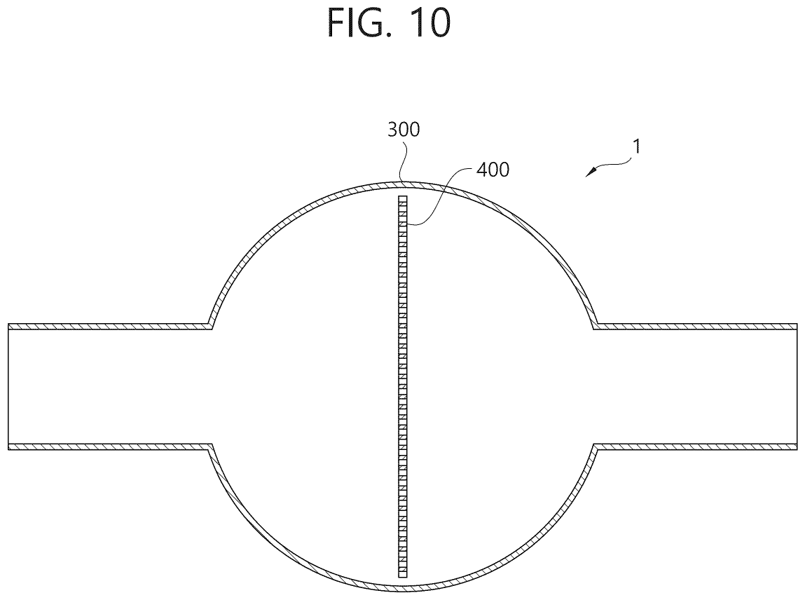

Because the shaft 510 of the rotor 500 extends to a length slightly smaller than the inner diameter of the bubble separating portion 300 as described above, the position of the rotor 500 may be maintained in the middle of the bubble separating portion 300. Further, the shaft 510 of the rotor 500 may be freely angled with respect to three axes. In this case, the center of gravity of the rotor 500 is located at a lower side in FIG. 10, so that the rotor 500 naturally stands up within the bubble separating portion 300.

Therefore, the rotor 500 in this embodiment is maintained standing up even though a patient changes the angle of his/her hand, thereby easily separating the bubbles 2000 from the injectable solution 1000 and collecting the separated bubbles 2000 in the bubble separating portion 300.

FIG. 10 is a cross-sectional view of a device 1 for separating bubbles 200 from an injectable drug according to a fifth embodiment of the disclosure.

Referring to FIG. 10, the device 1 for separating bubbles 200 from an injectable drug according to the fifth embodiment of the disclosure may include a mesh portion 400 placed inside the bubble separating portion 300. Specifically, the mesh portion 400 may be placed in a portion where the flow speed of the injectable solution 1000 is the lowest, i.e., a portion where the inner diameter is the largest, in the bubble separating portion 300. Because the injectable solution 1000 passes through the mesh portion 400 at the lowest flow speed, the bubbles 2000 may easily move escaping from the main flow direction toward the upper side of the bubble separating portion 300.

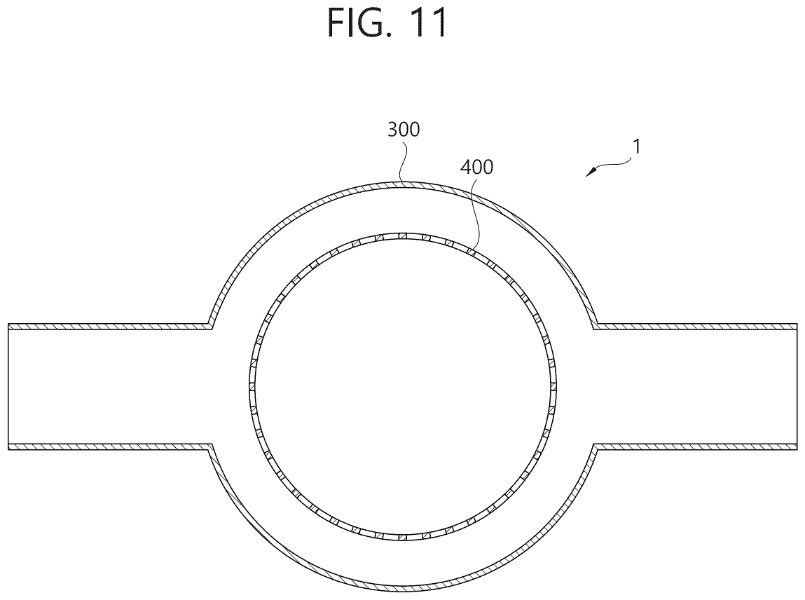

FIG. 11 is a cross-sectional view of a device 1 for separating bubbles 2000 from an injectable drug according to a sixth embodiment of the disclosure

Referring to FIG. 11, the device 1 for separating bubbles 200 from an injectable drug according to the sixth embodiment of the disclosure may include a mesh portion 400 shaped along the shell of a sphere. With this configuration, the bubbles 200 are easily separated from the main stream of the injectable solution 1000 without being affected by the angle of the device 1 for separating the bubbles 2000 from the injectable drug.

As described above, the device for separating bubbles from an injectable drug according to the disclosure can prevent the bubbles from entering the blood vessel when injecting the injectable solution into a vein. Therefore, the injectable solution is improved in stability when administered to the human body.

Further, the device for separating bubbles from an injectable drug according to the disclosure is easily added to a conventional infusion set or injectable solution set, thereby improving user convenience and efficiency.

According to the disclosure, the device for separating bubbles from an injectable drug can reliably separate bubbles from the injectable solution before the injectable solution is injected into a vein.

What is claimed is:

1. A device for separating bubbles from an injectable drug, comprising:
an inlet port configured to couple with a first tube;
an outlet port configured to couple with a second tube;
a bubble separating portion provided between the inlet port and the outlet port, and comprising a passage cross-sectional area more enlarged than the passage cross- sectional area of the first tube; and
a mesh portion provided in a passage of the inlet port or a passage of the bubble separating portion.

2. The device of claim 1, wherein the bubble separating portion comprises a spherical, ellipsoidal, or cylindrical inner space.

3. The device of claim 1, wherein a flow direction in the inlet port and a flow direction in the outlet port are parallel to each other.

4. The device of claim 3, wherein the bubble separating portion is rotationally symmetrical with respect to a central axis in the flow direction.

5. The device of claim 4, wherein the mesh portion comprises a honeycomb structure.
